(19) United States
(12) Patent Application Publication
(10) Pub. No.: US 2025/0177629 A1
(43) Pub. Date: Jun. 5, 2025

(54) COMPACT FLUID COLLECTION SYSTEMS

(71) Applicant: PUREWICK CORPORATION, Covington, GA (US)

(72) Inventors: Jonathan Robichaud, Decatur, GA (US); Rebecca Weiss, Atlanta, GA (US); Jill W. Jones, Avondale Estates, GA (US); Jason Jishen Cheng, Covington, GA (US)

(21) Appl. No.: 18/841,630

(22) PCT Filed: Feb. 28, 2022
(86) PCT No.: PCT/US2022/018159

§ 371 Date: Aug. 26, 2024

(51) Int. Cl. A61M 1/00; A61F 5/44; A61F 5/453; A61F 5/455

(52) U.S. Cl. CPC A61M 1/60; A61F 5/4401; A61F 5/453; A61F 5/4556; A61M 2202/0496; A61M 2210/1092; A61M 2210/167

(57) ABSTRACT

An example fluid collection system includes a fluid collection assembly. The fluid collection assembly includes a fluid impermeable barrier at least defining a chamber, at least one opening, and a fluid outlet. The fluid collection assembly also includes at least one porous material disposed in the chamber. The fluid collection system also includes a fluid storage container and a vacuum source that are both in fluid communication with the chamber of the fluid collection assembly.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

Embodiments are directed to fluid collection systems and methods of using the fluid collection systems. In an embodiment, a fluid collection system is disclosed. The fluid collection system includes a fluid collection assembly. The fluid collection assembly includes a fluid impermeable barrier at least defining a chamber, at least one opening, and a fluid outlet. The fluid collection assembly also includes at least one porous material disposed in the chamber. The fluid collection system also includes a fluid storage container and a vacuum source. The fluid collection assembly, the fluid storage container, and the vacuum source are in fluid communication with each other such that at least some of one or more bodily fluids received into the chamber and removed from the chamber and disposed into the fluid storage container using a suction provided by the vacuum source. At least one of the fluid impermeable barrier directly contacts at least one of the fluid storage container or the vacuum source or the fluid collection system further comprises at least one conduit exhibiting a length of about 50 cm or less connected to the fluid outlet of the fluid collection assembly and one of the fluid storage container or the vacuum source.

In an embodiment, a method of using a fluid collection system is disclosed. The method includes positioning at least one opening of a fluid collection assembly to be adjacent to a urethral opening of an individual or have a penis of the individual disposed through the at least one opening. The fluid collection assembly includes a fluid impermeable barrier defining at least a chamber, the at least one opening, and the fluid outlet. The fluid collection assembly also includes at least one porous material disposed in the chamber. The method also includes receiving one or more bodily fluids from the individual into the chamber and into the at least one porous material and applying a suction to the chamber via a vacuum source to remove at least some of the bodily fluids in the chamber and deposit the bodily fluids in a fluid storage container. At least one of the fluid impermeable barrier directly contacts at least one of the fluid storage container or the vacuum source or the fluid collection system further comprises at least one conduit exhibiting a length of about 50 cm or less connected to the fluid outlet of the fluid collection assembly and one of the fluid storage container or the vacuum source.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection system, according to an embodiment.

FIG. 1B is a schematic cross-sectional view of the fluid collection system taken along plane 1A-1A.

FIG. 2 is a schematic illustration of a fluid collection system, according to an embodiment.

FIG. 3 is a schematic view of a fluid collection system, according to an embodiment.

FIG. 4A is an isometric view of a fluid collection assembly, according to an embodiment.

FIG. 4B is a cross-sectional schematic of the fluid collection assembly taken along plane 4B-4B, according to an embodiment.

FIG. 5 is a cross-sectional view of a male fluid collection assembly according an embodiment.

FIG. 6 is a cross-sectional view of a fluid collection assembly, according an embodiment.

### DETAILED DESCRIPTION

Embodiments are directed to fluid collection systems and methods of using the fluid collection systems. An example fluid collection system includes a fluid collection assembly. The fluid collection assembly includes a fluid impermeable barrier at least defining a chamber, at least one opening, and a fluid outlet. The fluid collection assembly also includes at least one porous material disposed in the chamber. The fluid collection system also includes a fluid storage container and a vacuum source that are both in fluid communication with the chamber of the fluid collection assembly.

During use, the opening of the fluid collection assembly is positioned adjacent to a urethral opening (e.g., female urethral opening or urethral opening of a buried penis) or has a penis disposed through the opening. After positioning the fluid collection assembly, the individual using the fluid collection assembly may discharge urine or other bodily fluids (e.g., blood, sweat, etc.) into the chamber of the fluid collection assembly. The bodily fluids discharged into the chamber may be received into the porous material.

The bodily fluids may be removed from the chamber through the fluid outlet. Removal of the bodily fluids from the chamber may be facilitated by the vacuum source which is in fluid communication with the chamber via the fluid outlet. For example, the vacuum source may apply a suction to the chamber which pulls the bodily fluids towards the fluid outlet. The fluid storage container may also be in fluid communication with the chamber and the vacuum source such that the bodily fluids removed from the chamber may be deposited in and stored in the fluid storage container.

The fluid collection systems disclosed herein exhibit a compact size and/or is portable. The compact size and/or portability of the fluid collection systems disclosed herein is an improvement over conventional fluids collection systems. For example, the compact size and/or portability of the fluid collection systems disclosed herein allows the fluid collection systems disclosed herein to be used in embodiments where conventional fluid collection systems cannot be easily used and/or where conventional fluid collection systems cannot be used discretely.

An example of an embodiment where the compact size and/or portability of the fluid collection systems disclosed herein is an improvement over conventional fluid collection systems includes using the fluid collection systems disclosed herein with a wheelchair bound individual. There are several available options for a wheelchair bound individual to discharge bodily fluids. In an example, the individual may use restrooms having wheelchair accessible stalls in a restroom. However, use of wheelchair accessible stalls are limited by the availability of such stalls and require moving the individual from the wheelchair to the toilet and back again which may be difficult for some individuals (e.g., weakened or old individuals). An alternative to using wheelchair accessible stalls in restrooms includes using internal catheters (e.g., Foley catheters) that are configured to discharge into a toilet or into a drainage bag. However, such internal catheters requires specialized training and a clean environment (which may not be available in a public restroom) to inhibit urinary tract infections. Further, internal catheters that discharge into a drainage bag inherently include the drainage bag and relatively long conduits extending from the urethral opening of the individual to the drainage bag, both of which may be easily seen thereby preventing the internal catheter from being used discretely and cause embarrassment to the individual. An alternative to internal catheters includes using external catheters to receive bodily fluids discharged from the individual. However, such external catheters are often used with wall mounted vacuum sources or vacuum sources that need to be plugged in, both of which limit the areas in which the external catheters may be used. Portable vacuum sources may be used with external catheters. However, similar to internal catheters, the portable vacuum sources are configured to drain the bodily fluids into a drainage bag which requires relatively long conduits extending from the urethral opening to the drainage bag which prevent the external catheters from being used discretely. Further, the relatively long conduits and the drainage bag may also make intermittent use of such a fluid collection system difficult without leaking bodily fluids such that such fluid collection systems remain in place for prolonged periods of times (e.g., remain in place during multiple urinations).

The compact size and/or portability of the fluid collection systems disclosed herein resolve at least some of these issues. For example, the fluid collection systems may be used even when wheelchair accessible stalls are not available and have a lower risk of urinary tract infections compared to internal catheters. Also, the compact size and/or portability of the fluid collection systems disclosed herein allows the fluid collection systems disclosed herein to be used without drainage bags that are easily visible and may be used without conduits or with relatively short conduits (e.g., conduits exhibiting a length less than 80 cm or, more preferably, less than 50 cm). The compact size and/or portability of the fluid collection systems disclosed herein also allows such fluid collection systems to be used intermittently, such as used only when the individual is actively discharging bodily fluids and stored when the individual is in public.

It is noted that the fluid collection systems disclosed herein may be used in other embodiments than with wheelchair bound individuals. For example, the fluid collection systems disclosed herein may be used with individuals who are bed ridden, in hospital beds, or individuals who do not want to get out of bed to urinate during the night.

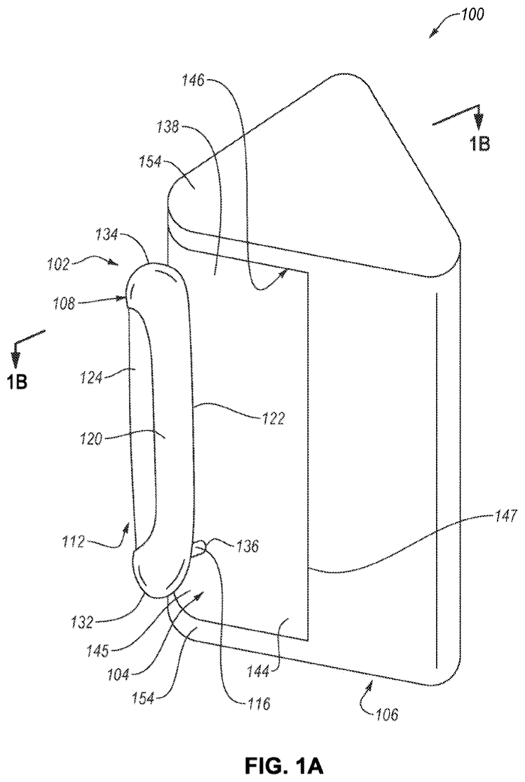

FIG. 1A is an isometric view of a fluid collection system 100, according to an embodiment. FIG. 1B is a schematic cross-sectional view of the fluid collection system 100 taken along plane 1A-1A. The fluid collection system 100 includes a fluid collection assembly 102, a fluid storage container 104, and a vacuum source 106. The fluid collection system 100 is configured such that the fluid collection assembly 102 directly contacts (e.g., is attached to) the fluid storage container 104 and the fluid storage container 104 directly contacts (e.g., is attached to) the vacuum source 106. Directly contacting the fluid collection assembly 102 against the fluid storage container 104 and directly contacting the fluid storage container against the vacuum source 106 causes the fluid collection system 100 to exhibit a small, compact size which, as will be discussed in more detail below, facilitates discrete operation of the fluid collection system 100.

The fluid collection assembly 102 is an example of a female fluid collection assembly for receiving and collecting bodily fluids from a female. The fluid collection assembly 102 includes a fluid impermeable barrier 108 defining a chamber 110, at least an opening 112, and a fluid outlet 114. The fluid collection assembly 102 also includes at least one porous material 124 disposed in a chamber 110. The fluid collection assembly 102 may further includes a conduit 116 is disposed through the fluid outlet 114 such that the conduit 116 is in fluid communication with the chamber 110.

The fluid impermeable barrier 108 at least partially defines a chamber 110 (e.g., interior region) and an opening 112. For example, the interior surface(s) 118 of the fluid impermeable barrier 108 at least partially defines the chamber 110 within the fluid collection assembly 102. The fluid impermeable barrier 108 temporarily stores the bodily fluids in the chamber 110. The fluid impermeable barrier 108 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, any other fluid impermeable material disclosed herein, or combinations thereof. As such, the fluid impermeable barrier 108 substantially prevents the bodily fluids from passing through the fluid impermeable barrier 108. In an example, the fluid impermeable barrier 108 may be air permeable and fluid impermeable. In such an example, the fluid impermeable barrier 108 may be formed of a hydrophobic material that defines a plurality of pores. At least one or more portions of at least an outer surface 120 of the fluid impermeable barrier 108 may be formed from a soft and/or smooth material, thereby reducing chaffing.

At least a portion of the outer surface 120 of the fluid impermeable barrier 108 may directly contact the fluid storage container 104. For example, the outer surface 120 may include a back surface 122 opposite the opening 112. At least a portion of the back surface 122 may directly contact the fluid storage container 104 which may prevent or at least inhibit the fluid storage container 104 and the vacuum source 106 from obstructing the opening 112 or otherwise preventing the opening 112 from being positioned adjacent to a urethral opening.

The opening 112 provides an ingress route for bodily fluids to enter the chamber 110. The opening 112 may be defined by the fluid impermeable barrier 108 such as by an inner edge of the fluid impermeable barrier 108. For example, the opening 112 is formed in and extends through the fluid impermeable barrier 108, from the outer surface 120 to the inner surface 118, thereby enabling bodily fluids to enter the chamber 110 from outside of the fluid collection assembly 102.

In some examples, the fluid impermeable barrier 108 may define a fluid outlet 114 sized to receive the conduit 116. The at least one conduit 116 may be disposed in the chamber 110 via the fluid outlet 114. The fluid outlet 114 may be sized and shaped to form an at least substantially fluid tight seal against the conduit 116 or the at least one conduit thereby substantially preventing the bodily fluids from escaping the chamber 110.

In an embodiment, the porous material 124 may be configured to wick any bodily fluids away from the opening 112, thereby preventing the bodily fluids from escaping the chamber 110. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the porous material 124, such as not include adsorption of the bodily fluids into the fluid permeable support 126. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the porous material 124 (e.g., absorbency), such as less than about 30 wt % of the dry weight of the porous material 124, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the porous material 124. The porous material 124 may also wick the bodily fluids generally towards an interior of the chamber 110, as discussed in more detail below. In an embodiment, the porous material 124 may include at least one absorbent or adsorbent material.

In an embodiment, the porous material 124 may include the fluid permeable membrane 128 disposed in the chamber 110. The fluid permeable membrane 128 may cover at least a portion (e.g., all) of the opening 112. The fluid permeable membrane 128 may be composed to wick the bodily fluids away from the opening 112, thereby preventing the bodily fluids from escaping the chamber 110.

In an embodiment, the fluid permeable membrane 128 may include any material that may wick the bodily fluids. For example, the fluid permeable membrane 128 may include fabric, such as a gauze (e.g., a silk, linen, or cotton gauze), another soft fabric, another smooth fabric, a nonwoven material, or any of the other porous materials disclosed herein. Forming the fluid permeable membrane 128 from gauze, soft fabric, and/or smooth fabric may reduce chaffing caused by the fluid collection assembly 102.

The fluid collection assembly 102 may include the fluid permeable support 126 disposed in the chamber 110. The fluid permeable support 126 is configured to support the fluid permeable membrane 128 since the fluid permeable membrane 128 may be formed from a relatively foldable, flimsy, or otherwise easily deformable material. For example, the fluid permeable support 126 may be positioned such that the fluid permeable membrane 128 is disposed between the fluid permeable support 126 and the fluid impermeable barrier 108. As such, the fluid permeable support 126 may support and maintain the position of the fluid permeable membrane 128. The fluid permeable support 126 may include any material that may wick, absorb, adsorb, or otherwise allow fluid transport of the bodily fluids, such as any of the fluid permeable membrane materials disclosed herein above. For example, the fluid permeable membrane material(s) may be utilized in a more dense or rigid form than in the fluid permeable membrane 128 when used as the fluid permeable support 126. The fluid permeable support 126 may be formed from any fluid permeable material that is less deformable than the fluid permeable membrane 128. For example, the fluid permeable support 126 may include a porous polymer (e.g., nylon, polyester, polyurethane, polyethylene, polypropylene, etc.) structure or an open cell foam, such as spun nylon fibers. In some examples, the fluid permeable support 126 may include a nonwoven material. In some examples, the fluid permeable support 126 may be formed from a natural material, such as cotton, wool, silk, or combinations thereof. In such examples, the material may have a coating to prevent or limit absorption of fluid into the material, such as a water repellent coating. In some examples, the fluid permeable support 126 may be formed from fabric, felt, gauze, or combinations thereof.

In some examples, the fluid permeable membrane 128 may be optional. For example, the porous material 124 may include only the fluid permeable support 126. In some examples, the fluid permeable support 126 may be optionally omitted from the fluid collection assembly 102. For example, the porous material 124 may only include the fluid permeable membrane 128.

In an embodiment, at least a portion of the porous material 124 (e.g., one or more of the fluid permeable membrane 128 or, more preferably, the fluid permeable support 126) may be hydrophobic. The porous material 124 may be hydrophobic when the porous material 124 exhibits a contact angle with water (a major constituent of bodily fluids) that is greater than about 90°, such as in ranges of about 90° to about 120°, about 105° to about 135°, about 120° to about 150°, about 135° to about 175°, or about 150° to about 180°. The hydrophobicity of the porous material 124 may limit absorption, adsorption, and solubility of the bodily fluids in the porous material 124 thereby decreasing the amount of bodily fluids held in the porous material 124. In an embodiment, at least a portion of the porous material 124 is hydrophobic or hydrophilic. In an embodiment, the fluid permeable support 126 is more hydrophobic (e.g., exhibits a larger contact angle with water) than the fluid permeable membrane 128. The lower hydrophobicity of the fluid permeable membrane 128 may help the porous material 124 receive the bodily fluids from the urethral opening while the hydrophobicity of the fluid permeable support 126 limits the bodily fluids that are retained in the porous material 124.

The porous material 124 may at least substantially completely fill the portions of the chamber 110 that are not occupied by the conduit 116. In some examples, the porous material 124 may not substantially completely fill the portions of the chamber 110 that are not occupied by the conduit 116. In such an example, the fluid collection assembly 102 includes the reservoir 130 disposed in the chamber 110.

The reservoir 130 is a substantially unoccupied portion of the chamber 110. The reservoir 130 may be defined between the fluid impermeable barrier 108 and one or both of the fluid permeable membrane 128 and fluid permeable support 126. The bodily fluids that are in the chamber 110 may flow through the fluid permeable membrane 128 and/or fluid permeable support 126 to the reservoir 130. The reservoir 130 may retain of the bodily fluids therein.

The bodily fluids that are in the chamber 110 may flow through the fluid permeable membrane 128 and/or fluid permeable support 126 to the reservoir 130. The fluid impermeable barrier 108 may retain the bodily fluids in the reservoir 130. While depicted in the distal end region 132, the reservoir 130 may be located in any portion of the chamber 110 such as the proximal end region 134. The reservoir 130 may be located in a portion of the chamber 110 that is designed to be located in a gravimetrically low point of the fluid collection assembly when the fluid collection assembly 102 is worn.

In some examples (not shown), the fluid collection assembly 102 may include multiple reservoirs, such as a first reservoir that is located at the portion of the chamber 110 closest to the inlet 136 of the conduit 116 (e.g., distal end region 132) and a second reservoir that is located at the portion of the of the chamber 110 that is at or near proximal end region 134). In another example, the fluid permeable support 126 is spaced from at least a portion of the conduit 116, and the reservoir 130 may be the space between the fluid permeable support 126 and the conduit 116.

The conduit 116 may be at least partially disposed in or otherwise in fluid communication with the chamber 110. The conduit 116 may be used to remove the bodily fluids from the chamber 110. The conduit 116 includes an inlet 136. The inlet 136 of the conduit 116 may be positioned adjacent to, within, or aft of the reservoir 130.

In an embodiment, as shown, the fluid outlet 114 is formed in or near the distal end region 132. In such an embodiment, the inlet 136 of the conduit 116 may be positioned within or proximate to the fluid outlet 114 which also positions the inlet 136 adjacent to or within the reservoir 130. In an embodiment, not shown, the conduit 116 may extend through a bore in the porous material 124 when the fluid outlet 114 is formed in or near the proximal end region 134.

The conduit 116 extends from the fluid collection assembly 102 to the fluid storage container 104 such that the fluid storage container 104 is in fluid communication with the fluid collection assembly 102. However, it is noted that, in some embodiments, the conduit 116 is omitted from the fluid collection system 100 since the fluid collection assembly 102 directly abuts the fluid storage container 104. In such embodiments, the fluid outlet 114 of the fluid collection assembly 102 is directly attached to a fluid inlet 136 of the fluid storage container 104. To facilitate directly attaching the fluid outlet 114 to the fluid inlet 136, the fluid outlet 114 and the fluid inlet 136 may be formed from materials that may form an interference fit with each other (e.g., non-flimsy materials) or may be attached together using an adhesive, weld (e.g., ultrasonic weld, radio frequency weld, etc.), another attachment technique, or combinations thereof.

The fluid storage container 104 includes one or more fluid impermeable walls 138. The fluid impermeable walls 138 of the fluid storage container 104 at least partially defines an enclosure 140 (e.g., interior region), the fluid inlet 136, and an outlet 142. The fluid storage container 104 stores the one or more bodily fluids removed from the chamber 110 of the fluid collection assembly 102 in the enclosure 140. In an embodiment, the enclosure 140 is substantially unoccupied to maximize the volume of bodily fluids that may be stored in the enclosure 140. In an embodiment, the enclosure 104 may include at least one porous material disposed therein that inhibits movement of the bodily fluids in the enclosure 140 and, when the porous material is hydrophilic, may inhibit bodily fluids leaving the enclosure 140.

The enclosure 140 of the fluid storage container 104 may exhibit a volume of about 100 milliliters (“mL”) to about 200 mL, about 150 mL to about 250 mL, about 200 mL to about 300 mL, about 250 mL to about 350 mL, about 300 mL to about 400 mL, about 350 mL to about 450 mL, about 400 mL to about 500 mL, about 450 mL to about 600 mL, about 500 mL to about 700 mL, about 600 mL to about 800 mL, about 700 mL to about 900 mL, about 800 mL to about 1 liter, about 900 mL to about 1.25 liters, or greater than 1 liter. The volume of the enclosure 140 depends on a variety of factors. Generally, it is desirable to minimize the volume of the enclosure 140 to minimize the size of the fluid collection system 100 which facilitates placement of at least a portion of the fluid collection system 100 under the clothing of the individual and/or between the legs of the individual. However, the volume of the enclosure 140 needs to exhibit a size that allows the enclosure 140 to receive substantially all of the bodily fluids discharged by the individual. Thus, the volume of the enclosure 140 may be selected based on balancing these two factors.

In an embodiment, the volume of the enclosure 140 may be selected based on the age and size of the individual. For instance, the volume of the enclosure 140 may be relatively small for a child (e.g., less than 200 ml) and may be relatively large for a large adult (e.g., about 500 ml or greater). In an embodiment, the volume of the enclosure 140 may be less than 500 ml when the fluid storage container 104 is disposable (discussed in more detail below) or is configured to be emptied after each discharge of bodily fluids while the volume of the enclosure 140 may be greater than 500 ml when the fluid storage container 104 is not disposable or configured to receive multiple urinations which may reduce waste and reduce the need to clean the fluid collection system 100. In an embodiment, the volume of the enclosure 140 may depend on whether the enclosure 140 is substantially empty or occupied by a porous material since the porous material may reduce the volume of bodily fluids that may be stored in the enclosure 140 for a given volume of the enclosure 140.

The fluid impermeable walls 138 may be formed from any fluid impermeable material disclosed herein or any other suitable fluid impermeable material. In an embodiment, the fluid impermeable walls 138 are formed from rigid material that exhibits sufficient stiffness that the fluid impermeable walls 138 are unlikely to collapse (e.g., maintain their shape) when a suction is provided to from the vacuum source 106 to the enclosure 140. The rigidity of the fluid impermeable walls 138 may ensure that air is present in the enclosure 140 when the enclosure 140 is not completely full of bodily fluids. The air in the enclosure 140 provides something for the vacuum source 106 to pull from the enclosure 140 other than bodily fluids since pulling the bodily fluids from the enclosure 140 into the vacuum source 106 may contaminate the vacuum source 106 (which may be difficult to clean) or may cause damage to the vacuum source 106 (e.g., short circuit the vacuum source 106). In an embodiment, the fluid impermeable walls 138 are formed from a flimsy material (i.e., a material that is likely to collapse when the suction from the vacuum source 106 is provided to the enclosure 140). Forming the fluid impermeable walls 138 from a flimsy material allows the fluid collection system 100 to exhibit as small of a volume as possible which may facilitate positioning of the fluid collection assembly 102.

The fluid impermeable walls 138 may include a front surface 145 and a back surface 147 opposite the front surface 145. At least a portion of the front surface 145 directly contacts the fluid collection assembly 102 and the back surface 147 may directly contact the vacuum source 106. Directly contacting the back surface 147 against the vacuum source 106 prevents or at least inhibits the vacuum source from obstructing the opening 112 of the fluid collection assembly 102 or inhibiting placement of the opening 112 against the urethral opening of the individual. However, it is noted that the vacuum source 106 may directly contact a lateral surface (e.g., a surface of the fluid impermeable walls 138 that is not opposite the front surface 145) of the fluid impermeable walls 138.

In an embodiment, the fluid impermeable walls 138 is directly attached to, either permanently or temporarily, to the fluid collection assembly 102 and/or the vacuum source 106. For example, the fluid impermeable walls 138 may be adhesively attached to, snapped to (e.g., using spring snaps), strapped to, or otherwise attached to the fluid collection assembly 102 and/or the vacuum source 106.

The fluid inlet 136 provides an ingress route for bodily fluids to enter the enclosure 140. The fluid inlet 136 extends through the fluid impermeable walls 138 thereby enabling the bodily fluids to enter the enclosure 140. In an embodiment, the fluid inlet 136 is formed in a portion of the fluid impermeable walls 138 that is adjacent to the fluid outlet 114 of the fluid collection assembly 102 which allows the fluid inlet 136 to be directly attached to the fluid outlet 114 or allows the conduit 116 extending from the fluid outlet 114 to the fluid inlet 136 to be as short as possible. For example, the fluid inlet 136 may be formed in or near a distal region 144 of the fluid storage container 104 when the fluid outlet 114 is formed in or near the distal end region 132 of the fluid collection assembly 102 and the fluid inlet 136 may be formed in or near a proximal region 146 of the fluid storage container 104 when the fluid outlet 114 is formed in or near the proximal end region 134 of the fluid collection assembly 102. In an embodiment, the fluid inlet 136 is formed in a portion of the fluid impermeable walls 138 that is not adjacent to the fluid outlet 114 of the fluid collection assembly 102. In such an embodiment, the conduit 116 may extend from the fluid outlet 114 to the fluid inlet 136, such as between the fluid collection assembly 102 and the fluid storage container 104 or adjacent to the portions of the fluid collection assembly 102 and the fluid storage container 104 that directly contact each other. Forming the fluid inlet 136 in a portion of the fluid storage container 104 that is spaced from the fluid outlet 114 may prevent backflow of the bodily fluids from the enclosure 140 to the chamber 110 when, for instance, the fluid outlet 114 is form in or near the distal end region 132.

In an embodiment, the fluid collection system 100 includes a first seal element 148 (schematically illustrated using a box in FIG. 1B). The first seal element 148 is configured to prevent backflow of the bodily fluids from the enclosure 140 to the chamber 110. In an embodiment, the first seal element 148 includes a check valve configured to allow bodily fluids to flow from the chamber 110 to the enclosure 140 while substantially preventing bodily fluids flowing from the enclosure 140 to the chamber 110. In such an embodiment, the suction provided by the vacuum source 106 may open the check valve but any backflow of the bodily fluids (e.g., caused by turning off the vacuum source 106) will cause the check valve to close. In an embodiment, the first seal element 148 includes a clamp. The clamp may be remove before the fluid collection assembly 102 receives the bodily fluids and the clamp may be reattached to the conduit 116 at least one of after the fluid collection assembly 102 stops receiving the bodily fluids, after substantially all of the bodily fluids are removed from the fluid collection assembly 102, or before the vacuum source 106 is turned off.

The outlet 142 proves an ingress route for a suction provided by the vacuum source 106 to be introduced into the enclosure 140 which, in turn, may cause the suction to be provided to the chamber 110. The outlet 142 extends through the fluid impermeable walls 138, thereby enabling the bodily fluids to enter the enclosure 140. In an embodiment, the outlet 142 is formed in a portion of the fluid impermeable walls 138 that is adjacent to a port 152 of the vacuum source which allows the outlet 142 to be directly attached to the port 152 or allows a conduit 116 extending from the outlet 142 to the port 152 to be as short as possible. In an embodiment, the outlet 142 is formed in a portion of the fluid impermeable walls 138 that is spaced from the port 152. In such an embodiment, the conduit 116 may extend from the outlet 142 to the port 152, such as between the fluid storage container 104 and the vacuum source 106 or adjacent to the portions of the fluid storage container 104 and the vacuum source 106 that directly contact each other.

In an embodiment, the outlet 142 of the fluid storage container 104 may be located at the proximal end 146 of the fluid storage container 104. The bodily fluids in the enclosure 140 are likely to pool at or near the distal region 144 of the fluid storage container 104. Positioning the outlet 142 in the proximal end 146 of the fluid storage container 104 decreases the likelihood that bodily fluids in the enclosure 140 reach the outlet 142. Generally, allowing the bodily fluids to reach the outlet 142 is discouraged since bodily fluids that reach the outlet 142 may flow into the vacuum source 106 which may contaminate the vacuum source 106 and/or damage the vacuum source. In an embodiment, the outlet 142 may be spaced from the proximal end 146 of the fluid storage container 104, for instance, when the outlet 142 or the vacuum source 106 includes a liquid impermeable (e.g., water impermeable) and gas permeable filter that prevents or at least inhibits bodily fluids flowing into the vacuum source 106.

In an embodiment, the fluid collection system 100 includes a second seal element 150 (schematically illustrated using a box in FIG. 1B). The second seal element 150 is configured to prevent flow of the bodily fluids from the enclosure 140 to the vacuum source 106. In an embodiment, the second seal element 150 includes a liquid impermeable (e.g., water impermeable) and gas permeable filter, such as a porous polytetrafluoroethylene filter. In such an embodiment, the liquid impermeable and gas permeable filter may allow the vacuum source 106 to provide suction to the enclosure 140, while preventing the bodily fluids from flowing from the enclosure to the vacuum source 106. In an embodiment, the second seal element 150 includes a clamp. The clamp may be remove before the fluid collection assembly 102 receives the bodily fluids and the clamp may be reattached to the conduit 116 at least one of after the fluid collection assembly 102 stops receiving the bodily fluids, after substantially all of the bodily fluids are removed from the fluid collection assembly 102, or before the vacuum source 106 is turned off. In an embodiment, the second sealing element 150 may include another type of seal (e.g., a check valve) that is configured to open when the suction is applied thereto, but closes when the suction is removed (e.g., turned off). In such an embodiment, the seal substantially prevents bodily fluids reaching the vacuum source 106 when the vacuum source 106 does not provide the suction (e.g., when the fluid collection system 100 is being carried around instead of being used).

The vacuum source 106 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 106 may provide a vacuum or suction to remove bodily fluids from the fluid collection assembly 102. In some examples, the vacuum source 106 may be powered by one or more of a power cord (e.g., connected to a power socket), one or more batteries, or even manual power (e.g., a hand operated vacuum pump). In some examples, the vacuum source 106 may be sized and shaped to fit outside of, on, or within the fluid collection assembly 102. For example, the vacuum source 106 may include one or more miniaturized pumps or one or more micro pumps. The vacuum sources disclosed herein may include one or more of a switch, a button, a plug, a remote, or any other device suitable to activate the vacuum source 106.

The vacuum source 106 may include one or more supports 154 that are configured to at least one of support the fluid storage container 104, help position the fluid storage container 104 when attaching the fluid storage container 104 to the vacuum source 106, or maintain the shape of the fluid storage container 104. In an embodiment, as shown, the support 154 may include a top panel and/or a bottom panel that extends from the rest of the vacuum source 106. The top panel and/or the bottom panel may be formed from a more rigid material than the fluid storage container 104 thereby allowing the supports 154 to help maintain the shape of the fluid storage container 104.

As will be discussed in more detail below, the fluid collection system 100 may be configured to at least partially fit between the urethral opening and clothing and/or between the thighs of the individual. The space between the urethral opening and the clothing and between the thighs of the individual may exhibit a generally triangular with the urethral opening of the individual at an apex of the generally triangular cross-sectional shape. As such, the fluid storage container 104 and the vacuum source 106 may exhibit a generally triangular cross-sectional shape at one or more locations thereof which may facilitate positioning the fluid storage container 104 and the vacuum source between the urethral opening and clothing and/or between the thighs of the individual. The fluid collection assembly 102 is attached to or otherwise extends from an apex of the generally triangular cross-sectional shape formed by the fluid storage container 104 and the vacuum source 106 so that the opening 112 of the fluid collection assembly 102 may be positioned adjacent to the urethral opening of the individual. Examples of shapes that exhibit a generally triangular cross-sectional shape include a generally triangular prism, triangular based pyramid, square based pyramid, or similar shapes with convex surfaces and/or rounded edges. It is noted that the fluid storage container 104 and the vacuum source 106 may exhibit non-generally triangular cross-sectional shapes, such as a generally circular cross-sectional shape, generally semi-circular cross-sectional shape, or a generally domed cross-sectional shape since these shapes may at least partially fit into the space between the urethral opening and the clothing and between the thighs of the individual.

As previously discussed, the fluid collection assembly 102 directly contacts the fluid storage container 104 and the fluid storage container 104 directly contacts the vacuum source 106. This configuration allows the fluid collection system 100 to exhibit a substantially compact size. The substantially compact size of the fluid collection system 100 allows the fluid collection system 100 to be at least partially positioned between the urethral opening of the individual and the clothing of the individual and/or between the legs of the individual. The compact size also allows the fluid collection system 100 to be intermittently used only when the individual is discharging bodily fluids. For example, the compact size allows the fluid collection system 100 to be positioned adjacent to the urethral opening of the individual during use and then moved away from the urethral opening when the individual is not discharging bodily fluids. The compact size of the fluid collection system 100 also allows for the fluid collection system 100 to be discretely stored in a bag or other container when not in use. Further, the compact size of the fluid collection system 100 also allows the fluid collection system 100 to be manipulated and handled using a single hand which leaves the other hand available to, for example, pull elastic pants away from the waist or hold a zipper open such that the fluid collection system 100 may be positioned with the opening 112 adjacent to the urethral opening of the individual.

During use, the individual may move to a private location, such as a restroom stall (not necessarily a wheelchair accessible stall), a bedroom closet, or other private location. It is noted that the individual does not necessarily need to move to private location but the private location may make using the fluid collection system 100 more discrete. The individual may then manipulate the clothing thereof to allow the fluid collection system 100 access to the urethral opening of the individual. For example, if the individual is wearing elastic pants and/or underwear (“elastic clothing”), the individual may pull the elastic clothing from the urethral opening with one hand and then move the fluid collection system 100 towards the urethral opening with the other hand such that the opening 112 is adjacent to the urethral opening. In such an example, it is noted that releasing the elastic clothing causes the elastic clothing to press against the fluid collection system 100 which, in turn, pushes the opening 112 of the fluid collection assembly 102 adjacent to the urethral opening. Pushing the opening 112 against the urethral opening decreases the likelihood that the bodily fluids discharged from the urethral opening leak into the individual's clothing or onto the individual's hands. Further, pushing the opening 112 against the urethral opening with the elastic clothing allows the individual to discharge the bodily fluids without the hands of the individual being close to the urethral opening which minimizes the likelihood that the hands become contaminated with bodily fluids and may make using the fluid collection system 100 more comfortable (i.e., the individual may find using the fluid collection system 100 less disgusting). In an example, if the individual is wearing pants with a zipper, the individual may unzip the zipper, optionally spread the zipper apart with one hand, and position the fluid collection system 100 with the other hand such that the opening 112 is adjacent to the urethral opening. In such an example, the individual may have to pull elastic underwear away from the urethral opening. In an example, if the individual is wearing relatively tight clothing, the individual may have to pull the clothing down to the individual's legs. It is noted that positioning the fluid collection system 100 adjacent to the urethral opening may be performed by a single individual without any specialized training. Positioning the fluid collection system 100 adjacent to the urethral opening may also be performed substantially without moving the lower body (especially if the individual is using elastic clothing or clothing with zippers) which is beneficial to individuals confined to a wheelchair or a bed.

The vacuum source 106 may be initially provided in an off state where no suction is provided. The vacuum source 106 may be switched to an on state before, during, or after positioning the fluid collection system 100. In an embodiment, the vacuum source 106 may include a button, switch, or other actuator that allows the vacuum source 106 to switch between the off and on states. In an embodiment, the vacuum source 106 may include one or more sensors (e.g., optical sensors, accelerometers, proximity sensors, etc.) that detects when the fluid collection system 100 is being positioned or is positioned adjacent to the urethral opening. The vacuum source 106 may switch from the off state to the on state when the sensors detect that the fluid collection system 100 is being positioned or is positioned adjacent to the urethral opening and may switch from the on state to the off state when the sensors detect that the fluid collection system 100 is being moves away from the urethral opening. In an embodiment, the vacuum source 106 may include one or more sensors that detects bodily fluids (e.g., moisture) in the chamber 110. In such an embodiment, the vacuum source 106 may switch from the off state to the on state when the sensors detect bodily fluids in the chamber 110 and may switch from the on state to the off state when the sensors detect substantially no moisture in the chamber 110.

After positioning the fluid collection system 100 and, optionally, switching the vacuum source 106 to the on state, the individual may discharge bodily fluids through the opening 112, into the chamber 110, and into the porous material 124. The suction provided from the vacuum source 106 to the enclosure 140 and the chamber 110 may move the bodily fluids towards to the fluid outlet 114 and remove the bodily fluids from the chamber 110 to the enclosure 140.

In an embodiment, at least one of the fluid collection assembly 102 or the fluid storage container 104 is disposable (e.g., configured for single use). Disposing of the fluid collection assembly 102 and/or the fluid storage container 104 reduces the amount of cleaning of the fluid collection system 100 that is needed after discharging the fluid collection system 100. The fluid collection assembly 102 and/or the fluid storage container 104 are disposable when the fluid collection assembly 102 and/or the fluid storage container 104 may be detached from the vacuum source 106 without damaging the fluid collection assembly 102, the fluid storage container 104, and the vacuum source 106 and substantially without causing the bodily fluids to leak from the fluid storage container 104. The fluid collection assembly 102 and/or the fluid storage container 104 is disposable, for example, when the fluid collection assembly 102 is attached to the fluid storage container 104 and/or the fluid storage container 104 is attached to the vacuum source 106, respectively, using a weak adhesive, snaps, a strap, or an interference fit. When at least one of the fluid collection assembly 102 or the fluid storage container 104 are disposable, the method of using the fluid collection system 100 may include detaching the fluid collection assembly 102 from the fluid storage container 104 and/or the fluid storage container 104 from the vacuum source 106 and disposing of the fluid collection assembly 102 and/or the fluid storage container 104 after the individual discharges the bodily fluids into the chamber 110. In some examples, the method may also include using a seal element (e.g., a clamp, etc.) to prevent bodily fluids from leaking from the enclosure 104 before detaching the fluid collection assembly 102 from the fluid storage container 104 or the fluid storage container 104 from the vacuum source 106 to prevent bodily fluids from leaking from the fluid storage container 104. After disposing of the fluid collection assembly 102 and/or the fluid storage container 104, a replacement fluid collection assembly and/or replacement fluid storage container may be added to the fluid collection system 100 thereby allowing the fluid collection system 100 to be reused. The replacement fluid collection assembly and/or the replacement fluid storage container may be the same as or different than the original fluid collection assembly 102 and/or the original fluid storage container 104.

Regardless if the fluid collection assembly 102 and/or the fluid storage container 104 are disposable, the vacuum source 106 and any component that remains attached to the vacuum source 106 may need to be cleaned after the individual discharged the bodily fluids. Cleaning the remaining components of the fluid collection system 100 may include wiping such components with a wipe (e.g., disinfecting wipe), flowing sterilizing fluids through any portion of the remaining components that may have had bodily fluids flowing therethrough, or otherwise cleaning such components.

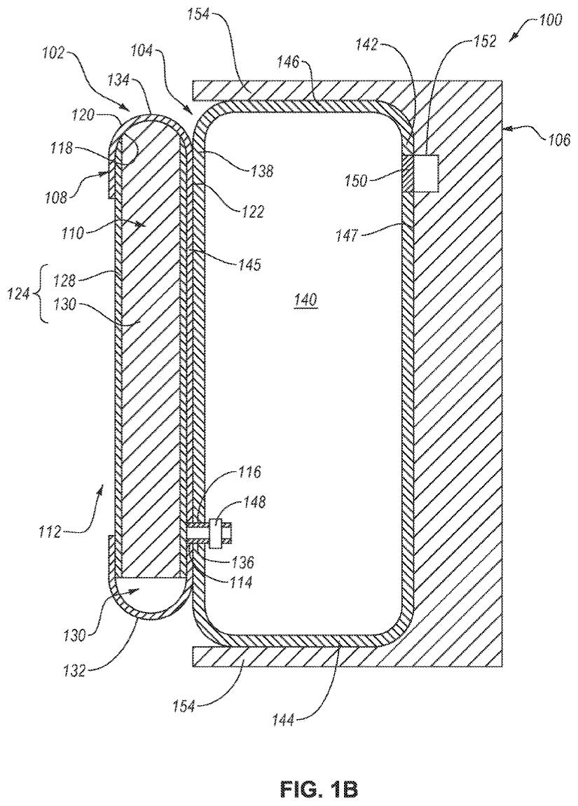

The fluid collection system 100 includes the fluid collection assembly 102 positioned upstream from the fluid storage container 104 and the fluid storage container 104 positioned upstream from the vacuum source 106. However, it is noted that the fluid collection systems disclosed herein may have different arrangements. For example, FIG. 2 is a schematic illustration of a fluid collection system 200, according to an embodiment. Except as otherwise disclosed herein, the fluid collection system 200 is the same as or substantially similar to any of the fluid collection systems disclosed herein. For example, the fluid collection system 200 includes a fluid collection assembly 202, a fluid storage container 204, and a vacuum source 206.

The components of the fluid collection system 200 are arranged such that the fluid collection assembly 202 is positioned upstream from the vacuum source 206 and the vacuum source 206 is positioned upstream from the fluid storage container 204 (e.g., the fluid storage container 204 is downstream from the vacuum source 206). This allows the fluid collection assembly 202 and the fluid storage container 204 to directly contact (e.g., be directly attached to) the vacuum source 206. In an example, the vacuum source 206 may be more rigid than the fluid collection assembly 202 and the fluid storage container 204. As such, directly contacting the fluid collection assembly 202 and the fluid storage container 204 to the vacuum source 206 allows the vacuum source 206 to provide structure and support to the fluid collection assembly 202 and the fluid storage container 204. In an example, when the fluid collection system 200 is at least partially positioned between the thighs of the individual, positioning the fluid storage container 204 downstream from the vacuum source 206 positions the fluid storage container 204 further from the urethral opening of the individual than if the fluid storage container 204 was positioned upstream from the vacuum source 206. The space between the thighs increases with increasing distance from the urethral opening. Thus, positioning the fluid storage container 204 downstream from the vacuum source 206 (e.g., further from the urethral opening) provides a larger space for the fluid storage container 204 to be positioned thereby allowing the enclosure of the fluid storage container 204 to exhibit a larger volume. In an example, positioning the fluid storage container 204 downstream from the vacuum source 206 allows the fluid storage container 204 to be formed from a more flimsy material than if the fluid storage container 204 is positions upstream from the vacuum source 206 since the fluid storage container 204 does not support the fluid collection assembly 202.

In the illustrated embodiment, the vacuum source 206 includes a first port that is in fluid communication with the fluid outlet of the fluid collection assembly 202 and the vacuum source 206 includes a second port that is in fluid communication with the inlet of the fluid storage container 204.

In an embodiment, the vacuum source 206 is a peristatic pump since such a pump may limit contamination of the vacuum source 206 with the bodily fluids. The peristatic pump includes a compressible tube and the bodily fluids flowing through the vacuum source 206 are limited to the compressible tube as the bodily fluids flow from the first port to the second port of the vacuum source 206. Limiting contamination of the vacuum source 206 by the bodily fluids to the compressible tube may facilitate cleaning of the vacuum source 206 (e.g., the vacuum source 206 may be cleaned by merely flowing a fluid, such as a sterilizing fluid, through the compressible tube) and prevents damage to the rest of the vacuum source 206. In an embodiment, the vacuum source 206 may include any other suitable vacuum source, such as any of the vacuum sources disclosed herein.

The fluid collection systems illustrated in FIGS. 1A-2 illustrate the fluid collection assemblies, the fluid storage containers, and the vacuum sources directly contacting each other which increases the compactness of the fluid collection systems.

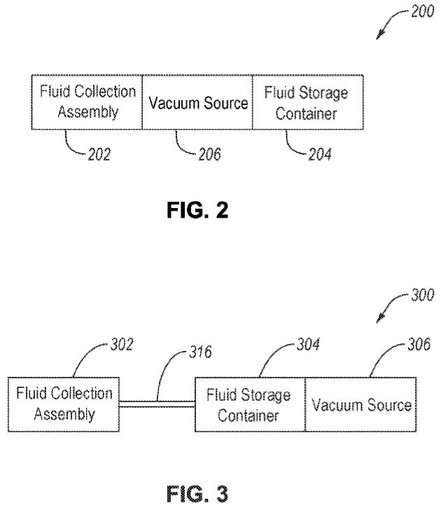

However, in some embodiments, one or more of the fluid collection assembly, the fluid storage container, of the vacuum source of any of the fluid collection systems disclosed herein may be spaced from each other by at least one conduit. For example, FIG. 3 is a schematic view of a fluid collection system 300, according to an embodiment. Except as otherwise disclosed herein, the fluid collection system 300 may be the same as or substantially similar to any of the fluid collection systems disclosed herein. For example, the fluid collection system 300 includes a fluid collection assembly 302, a fluid storage container 304, and a vacuum source 306.

In the illustrated embodiment, the fluid collection assembly 302 is spaced from the fluid storage container 304 and the vacuum source 306 by at least one conduit 316. The conduit 316 provides additional flexibility when positioning the fluid collection assembly 302 adjacent to the urethral opening of the individual. The conduit 316 also allows the fluid collection assembly 302 to be positioned adjacent to the urethral opening while the fluid storage container 304 and the vacuum source 306 are not positioned between the urethral opening and the clothes of the individual or between the thighs of the individual.

Spacing the fluid storage container 304 and the vacuum source 306 from locations that are not positioned between the urethral opening of the individual and the clothes of the individual or between the thighs of the individual allows the fluid storage container 304 and the vacuum source 306 to exhibit shapes and/or sizes that may make it difficult to position the fluid storage container 304 and the vacuum source 306 between the urethral opening of the individual and the clothes of the individual or between the thighs of the individual. However, the fluid storage container 304 and the vacuum source 306 may exhibit any of the sizes discussed above since the sizes discussed above may facilitate handling of and discretely storing the fluid storage container 304 and the vacuum source 306. The fluid storage container 304 and the vacuum source 306 may also exhibit any of the sizes and shapes discussed above to allow the individual to position the fluid storage container 304 and the vacuum source 306 between the urethral opening and the clothes of the individual and/or between the thighs of the individual if desired.

The conduit 316 allows the fluid collection assembly 302 to be spaced from the fluid storage container 304 and the vacuum source 306 by a maximum distance d. The maximum distance d may be selected to be about 1 cm to about 5 cm, about 2.5 cm to about 7.5 cm, about 5 cm to about 10 cm, about 7.5 cm to about 12.5 cm, about 10 cm to about 15 cm, about 12.5 cm to about 17.5 cm, about 15 cm to about 20 cm, about 17.5 cm to about 22.5 cm, about 20 cm to about 25 cm, about 22.5 cm to about 30 cm, about 25 cm to about 35 cm, about 30 cm to about 40 cm, about 35 cm to about 45 cm, about 40 cm to about 50 cm, about 45 cm to about 55 cm, about 50 cm to about 60 cm, about 55 cm to about 65 cm, about 60 cm to about 70 cm, about 65 cm to about 75 cm, or about 70 cm to about 80 cm. Generally, it is desirable to minimize the maximum distance d to make it easier to discretely handle the fluid collection system 300, make cleaning the fluid collection system 300 easier, and make it easier to store the fluid collection system 300. However, it is noted that the maximum distance d may be selected to be sufficiently large to allow the fluid storage container 304 and the vacuum source 306 to not be positioned between the urethral opening and the clothes of the individual and/or between the thighs of the individual when such a position of the fluid storage container 304 and the vacuum source 306 is desired. It is noted that the maximum distance d disclosed herein may be too small for conventional fluid collection assemblies since the maximum distance d disclosed herein is too small, especially when the maximum distance d is less than 50 cm, to extend from a device that receives urine (e.g., Foley catheter) to a conventionally used drainage bag (e.g., a drainage bag hanging from a bend, the back of a wheelchair, or under the wheelchair seat) or conventionally used vacuum source (e.g., a vacuum source on a nightstand or a wall-mounted vacuum source).

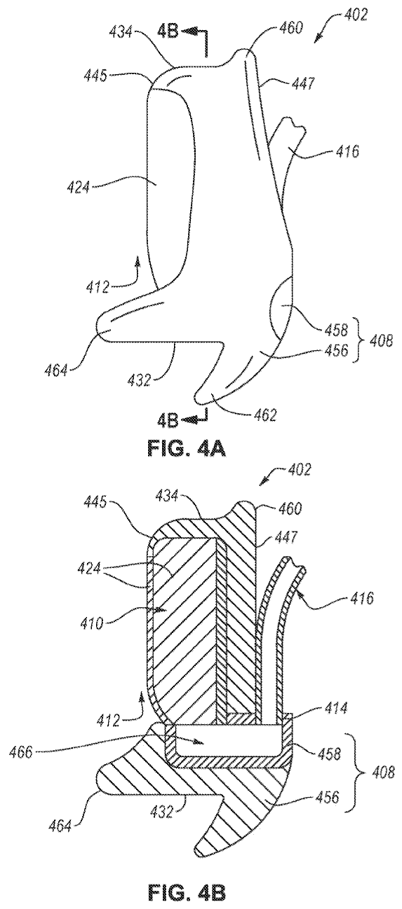

The fluid collection assembly 102 illustrated in FIGS. 1A and 1B is merely one example of a fluid collection assembly that may be used in the fluid collection systems disclosed herein. Further examples of fluid collection assemblies that may be used in the fluid collection systems disclosed are illustrated and discussed in relation to FIGS. 4A-6. Except as otherwise disclosed herein, the fluid collection assemblies of FIGS. 4A-6 are the same as or substantially similar to any of the fluid collection assemblies disclosed herein and may be used in any of the fluid collection systems disclosed herein.

FIG. 4A is an isometric view of a fluid collection assembly 402, according to an embodiment. FIG. 4B is a cross-sectional schematic of the fluid collection assembly 402 taken along plane 4B-4B, according to an embodiment. The fluid collection assembly 402 is example of a female fluid collection assembly for receiving and collecting bodily fluids from a female. The fluid collection assembly 402 includes a fluid impermeable barrier 408 that is the same or similar to any of the fluid impermeable barriers disclosed herein in one or more aspects. The fluid impermeable barrier 408 at least defines a chamber 410, at least one opening 412, and a fluid outlet 414. The fluid collection assembly 402 also includes at least one porous material 424 positioned within the chamber 410. The porous material 424 may be the same or similar to any of the porous materials disclosed herein in one or more aspects. In an embodiment, as illustrated, the fluid collection assembly 402 may also include at least one conduit 416 partially positioned within the fluid outlet 414 that is configured to remove one or more bodily fluids from the chamber 410. The conduit 416 may not extend through the porous material 424. In an embodiment, the conduit 416 may be omitted when the fluid outlet 414 is directly attached to the fluid inlet of the fluid storage container (not shown) or the port of the vacuum source (not shown).

In an embodiment, the fluid impermeable barrier 408 may include a shell 456 and a connector piece 458 secured to the shell 456. The shell 456 of the fluid collection assembly 402 includes a proximal end region 434, a distal end region 432 opposite the proximal end region 434, a front side 445, and a back side 447 opposite the front side 445. Generally, during use, the distal end region 432 is closer to the gluteal cleft of the individual than the proximal end region 434 and the front side 445 generally faces the vaginal region of the individual. The shell 456 may be formed from silicone, neoprene, a thermoplastic elastomer, or other fluid impermeable material.

In an embodiment, the shell 456 includes one or more flanges. The flanges may provide more locations for underwear or other clothing to contact and press against the fluid collection assembly 402 which may facilitate securing the fluid collection assembly 402 to the vaginal region of the individual and may improve patient comfort. In an embodiment, the flanges may include at least one of an upper flange 460 forming the proximal end region 434 and a bottom flange 462 opposite the upper flange 460 that forms the distal end region 432.

The flanges of the body may extend from the rest of the shell 456 by a distance that is about 1 mm or greater, about 1 mm or greater, about 3 mm or greater, about 4 mm or greater, about 5 mm or greater, about 6 mm or greater, about 7.5 mm or greater, about 1 cm or greater, about 1.25 cm or greater, about 1.5 cm or greater, about 1 cm or greater, about 1.5 cm or greater, about 3 cm or greater, about 4 cm or greater, about 5 cm or greater, or in ranges of about 1 mm to about 3 mm, about 1 mm to about 4 mm, about 3 mm to about 5 mm, about 4 mm to about 6 mm, about 5 mm to about 7.5 mm, about 6 mm to about 1 cm, about 7.5 mm to about 1.25 cm, about 1 cm to about 1.5 cm, about 1.25 cm to about 1 cm, about 1.5 cm to about 1.5 cm, about 1 cm to about 3 cm, about 1.5 cm to about 4 cm, or about 3 cm to about 5. The distance that the flanges extend from the rest of the shell 456 may be selected based on the expected size of the vaginal region of the individual (e.g., larger flanges for a larger vaginal region) or the expect rotational forces applied to the fluid collection assembly 402 during use. In some examples, at least some of the flanges may extend further from the rest of the shell 456 that other flanges. For instance, as illustrated, the bottom flange 462 may extend further from the rest of the fluid impermeable barrier 408 than the upper flange 460 since some individuals may find the longer bottom flange 462 more comfortable.

In an embodiment, the one or more flanges may exhibit a concave curve relative to the front side 445 of the shell 456. The concave curve of the flanges may extend from the proximal end region 434 to the distal end region 432. The concave curve of the flanges may allow the flanges to better conform to the shape of the vaginal region since the vaginal region is curved. Conforming the flanges to the shape of the vaginal region may make the fluid collection assembly 402 more comfortable by more uniformly distributing pressure across the vaginal region, especially when the flanges contact the labia majora

In an embodiment, the shell 456 may include a sump 464 at or near the distal end region 432. The sump 464 may extend outwardly from the front side 445 of the shell 456. During use, the sump 464 is configured to be at, near, or otherwise in fluid communication with a gravimetric low point of the porous material 424. For example, the sump 464 may receive a portion of the porous material 424 therein. The sump 464 may receive at least some of the bodily fluids that are received by the porous material 424. The sump 464 may prevent or at least inhibit bodily fluids from leaking from the fluid collection assembly 402. The sump 464 may include at least a portion of the connector piece 458 at least partially disposed therein.

In an embodiment, the shell 456 may define a recess that is configured to receive a conduit 416. The recess may extend from or near the proximal end region 434 to or near the distal end region 432 thereby allowing the conduit 416 to extend from or near the individual's abdominal region to the connector piece 458. In an embodiment, the recess may be configured such that the shell 456 encloses and/or abuts less than 50% of a circumference of the conduit 416, thereby allowing the conduit 416 to freely enter and leave the recess during use. Allowing the conduit 416 to freely enter and leave the recess may facilitate positioning of the fluid collection assembly 402 such that the porous material 424 is adjacent to the vaginal region even when the conduit 416 is bending away from the vaginal region. Also, allowing the conduit 416 to freely enter and leave the recess may increase the likelihood that movement of the conduit 416 does not move the porous material 424 relative to the vaginal region since movement of the porous material 424 may cause leaking. In an embodiment, at least a portion of the recess may be configured such that the shell 456 encloses and/or abuts more than 50% (e.g., 51% to about 55%, about 53% to about 57%, or about 55% to about 60%) of the circumference of the conduit 416. Enclosing more than 50% of the circumference of the conduit 416 may more securely attach the conduit 416 to the shell 456 and may allow the conduit 416 to provide additional structure to the shell 456. The percentage of the conduit 416 enclosed and/or abutted by the shell 456 may be selected such that the inherent elasticity of the shell 456 and the conduit 416 allows the conduit 416 to be easily snapped into and out of the recess. As such, the conduit 416 may be removed from the recess to facilitate positioning the porous material 424 adjacent to the vaginal region or when the conduit 416 is moved.

As previously discussed, the fluid impermeable barrier 408 includes a connector piece 458 that is attached to (e.g., with an adhesive, welding, interference fit, etc.) or integrally formed with the shell 456. The connector piece 458 is positioned at or near the distal end region 432 of the shell 456 which allows the connector piece 458 to receive bodily fluids that flow to the gravimetric low point of the porous material 424. In an embodiment, a portion of the connector piece 458 may be positioned in the sump 464 of the shell 456. In an embodiment, not shown, the connector piece 458 may form the sump 464 instead of the shell 456.

The connector piece 458 is configured to be connected to the conduit 416. As such, the connector piece 458 may define the fluid outlet 414 which configured to be attached to or otherwise in fluid communication with the conduit 416. The fluid outlet 414 may be positioned adjacent to the back side 447 of the fluid impermeable barrier 408. The connector piece 458 may also define a channel 466 (e.g., conduit) configured to allow the porous material 424 and the sump 464 to be in fluid communication with the conduit 416.

In an embodiment, at least a portion of the connector piece 458 may exhibit a rigidity that is greater than the shell 456. The increased rigidity of the connector piece 458 relative to the shell 456 may facilitate attachment of the conduit 416 to the connector piece 458. In an example, the connector piece 458 may exhibit a rigidity that is greater than the shell 456 when the connector piece 458 is formed from a material exhibiting at least one of a greater Young's modulus (i.e., modulus of elasticity), yield strength, or ultimate tensile strength than a material that forms the shell 456. In an example, the connector piece 458 may exhibit a rigidity that is greater than the shell 456 when the connector piece 458 exhibits a thickness that is greater than the shell 456.

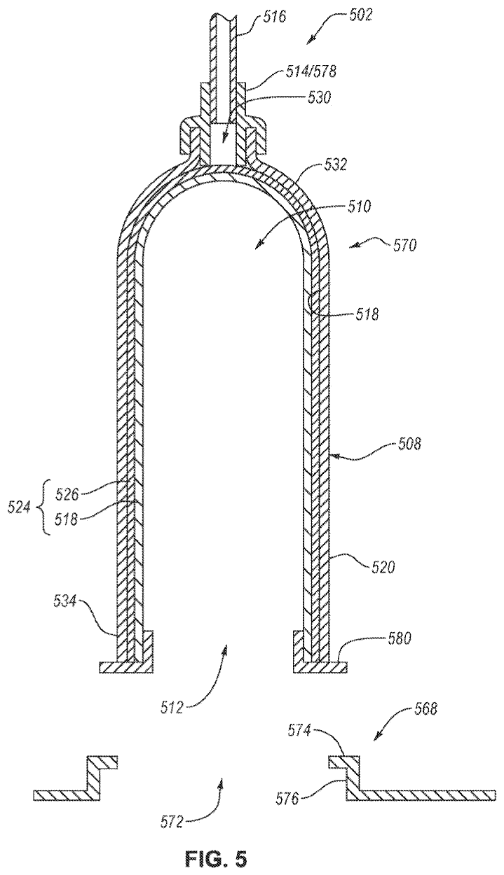

The fluid collection assemblies shown in FIGS. 1A, 1B, 4A, and 4B are examples of female fluid collection assemblies that are configured to collect bodily fluids from females (e.g., collect urine from a female urethra). However, the fluid collection assemblies, systems, and methods disclosed herein may include male fluid collection assemblies shaped, sized, and otherwise configured to collect bodily fluids from males (e.g., collect urine from a male urethra). FIG. 5 is a cross-sectional view of a male fluid collection assembly 500 according an embodiment.

The fluid collection assembly 502 includes a base 568 (e.g., annular base) and a sheath 570. The base 568 is sized, shaped, and made of a material to be coupled to skin that surrounds the male urethral opening (e.g., penis) and have the male urethral opening positioned therethrough. For example, the base 568 may define an aperture 572. The base 568 is sized and shaped to be positioned around the male urethral opening (e.g., positioned around and/or over the penis) and the aperture 572 may be configured to have the male urethral opening positioned therethrough. The base 568 may also be sized, shaped, made of a material, or otherwise configured to be coupled (e.g., adhesively attached, such as with a hydrogel adhesive) to the skin around the male urethral opening (e.g., around the penis). In an example, the base 568 may exhibit the general shape or contours of the skin surface that the base 568 is selected to be coupled with. The base 568 may be flexible thereby allowing the base 568 to conform to any shape of the skin surface. The base 568 may include a laterally (e.g., radially) extending flange 574. The base 568 also defines a hollowed region that is configured to receive (e.g., seal against) the sheath 570. For example, the base 568 may include a longitudinally extending flange 576 that extends upwardly from the base 568. The longitudinally extending flange 576 may be tall enough to prevent the sheath 570 from being accidentally removed from the base 568 (e.g., at least 0.25 cm tall, 1 cm tall, at least 3 cm tall, or at least 5 cm tall). The base 568 is located at a proximal end region 534 (with respect to a wearer) of the fluid collection assembly 502.

The sheath 570 includes (e.g., may be formed from) a fluid impermeable barrier 508 that is sized and shaped to fit into the hollowed region of the base 568. For example, the sheath 570 may be generally tubular or cup-shaped, as shown. The generally tubular or cup-shaped fluid impermeable barrier 508 may at least partially define the outer surface 520 of the sheath 570. The fluid impermeable barrier 508 may be similar or identical to and of the fluid impermeable barriers disclosed herein, in one or more aspects. For example, the fluid impermeable barrier 508 may be constructed of any of the materials disclosed herein for the fluid impermeable barrier. The fluid impermeable barrier 508 at least partially defines the chamber 510. For example, the inner surface 518 of the fluid impermeable barrier 508 at least partially defines the perimeter of the chamber 510. The chamber 510 may at least temporarily retain bodily fluids therein. As shown, the fluid collection assembly 502 may include the porous material 524 therein. The porous material 524 may be similar or identical any of the porous materials disclosed herein, in one or more aspects. For example, the porous material 524 may include one or more of a fluid permeable membrane 528 or a fluid permeable support 526. The fluid impermeable barrier 508 may also define an opening 512 extending through the fluid impermeable barrier 508 that is configured to have a male urethral opening positioned therethrough.

The sheath 570 also may include at least a portion of the conduit 516 therein, such as at least partially disposed in the chamber 510. For example, the conduit 516 may extend from the sheath 570 at the distal end region 532 to a proximal end region 534 at least proximate to the opening 512. The proximal end region 534 may be disposed near or on the skin around the male urethral opening (e.g., on the penis or pubic area therearound). Accordingly, when an individual lays on their back, bodily fluids (e.g., urine) may aggregate near the opening 512 against the skin of the subject. The bodily fluids may be removed from the chamber 510 via the conduit 516. In an embodiment, the conduit 516 may be omitted, for example, when the fluid outlet 514 is directly connected to the fluid inlet of the fluid storage container (not shown) or the port of the vacuum source (not shown).

In some examples, the fluid impermeable barrier 508 may be constructed of a material and/or have a thickness that allows the sheath 570 to collapse when placed under vacuum, such as to remove air around a penis in the fluid collection assembly 502 during use. In such examples, the conduit 516 may extend only to or into the distal end region 532 in the chamber 510 (e.g., not through to the area adjacent the opening 512). In such examples, urine may be collected and removed from the fluid collection assembly 502

In an example, portions of the chamber 510 may be substantially empty due to the varying sizes and rigidity of the male penis. However, in some examples, the outermost regions of the chamber 510 (e.g., periphery of the interior regions of the sheath 570) may include the porous material 524 (e.g., one or more of the fluid permeable membrane 528 and fluid permeable support 526). For example, the porous material 524 may be bonded to the inner surface 518 of the fluid impermeable barrier 508. The porous material 524 may be positioned (e.g., at the distal end of the chamber 510) to blunt a stream of urine from the male urethral opening thereby limiting splashing and/or to direct the bodily fluids to a selected region of the chamber 510. Since the chamber 510 is substantially empty (e.g., substantially all of the chamber 510 forms a reservoir), the bodily fluids are likely to pool at a gravimetrically low point of the chamber 510. The gravimetrically low point of the chamber 510 may be at an intersection of the skin of an individual and the fluid collection assembly 502, a corner formed in the sheath 570, or another suitable location depending on the orientation of the wearer.

The porous material 524 may include one or more of the fluid permeable membrane 528 or the fluid permeable support 526. The fluid permeable membrane 528 and the fluid permeable support 526 may be similar or identical to any of the fluid permeable membranes and the fluid permeable supports, respectively disclosed herein, in one or more aspects. One or more of the fluid permeable membrane 528 or the fluid permeable support 526 may be disposed between the fluid impermeable barrier 508 and a penis inserted into the chamber 510. The fluid permeable membrane 528 may be positioned between the fluid impermeable barrier 508 and a penis inserted into the chamber 510, such as between the fluid permeable support 526 and penis of a wearer as shown. The fluid permeable support 526 may be positioned between the fluid permeable membrane 528 and the fluid impermeable barrier 508. The inner surface 518, optionally including the end of the chamber 510 substantially opposite the opening 512, may be covered with one or both the fluid permeable membrane 528 or the fluid permeable support 526. The fluid permeable support 526 or the fluid permeable membrane 528 may be affixed (e.g., adhered) to the fluid impermeable barrier 508. The fluid permeable support 526 or the fluid permeable membrane 528 may be affixed to each other. In some examples, the porous material 524 only includes the fluid permeable membrane 528 or the fluid permeable support 526.

The fluid collection assembly 502 includes a cap 578 at a distal end region 532. The cap 578 defines an interior channel through which the bodily fluids may be removed from the fluid collection assembly 502. The interior channel is in fluid communication with the chamber 510. The cap 578 may be disposed over at least a portion of the distal end region 532 of one or more of the fluid impermeable barrier 508 or the porous material 524. The cap 578 may be made of a polymer, rubber, or any other fluid impermeable material. The cap 578 may be attached to one or more of the fluid impermeable barrier 508, the porous material 524, or the conduit 516. The cap 578 may cover at least a portion of the distal end region 532 of the fluid collection assembly 502.

The cap 578 may define a fluid outlet 514 that is sized and configured to receive and fluidly seal against the conduit 516. The conduit 516 may extend a distance within or through the cap 578, such as to the porous material 524, through the porous material 524, or to a point set-off from the porous material 524. In the latter example, as depicted in FIG. 5B, the interior channel of the cap 578 may define a reservoir 530 therein.

The reservoir 530 is an unoccupied portion of device such as in the cap 578 and is void of other material. In some examples, the reservoir 530 is defined at least partially by the porous material 524 and the cap 578. During use, the bodily fluids that are in the chamber 510 may flow through the porous material 524 to the reservoir 530. The reservoir 530 may store at least some of the bodily fluids therein and/or position the bodily fluids for removal by the conduit 516. In some examples, at least a portion of the porous material 524 may extend continuously between at least a portion of the opening of the interior channel and chamber 510 to wick any bodily fluids from the opening directly to the reservoir 530.

In some examples (not shown), the fluid impermeable barrier 508 may be disposed on or over the cap 578, such as enclosing the cap 578 within the chamber 510.

In some examples, the sheath 570 may include at least a portion of the conduit 516 therein, such as at least partially disposed in the chamber 510. For example, the conduit 516 may extend from the sheath 570 to a region at least proximate to the opening 512. The inlet of the conduit 516 may be positioned adjacent to the annular base 580. The inlet of the conduit 516 may be positioned to be adjacent or proximate to the gravimetrically low point of the chamber 510, such as adjacent to the annular base 580. For example, the inlet may be co-extensive with or offset from the opening 512. In examples, the inlet may be positioned adjacent to the distal end region 532 of the sheath 570 (e.g., substantially opposite the opening 512).

The proximal end region 534 may be disposed near or on the skin around the male urethral opening (e.g., around the penis) and the inlet of the conduit 516 may be positioned in the proximal end region 534. The outlet of the conduit 516 may be directly or indirectly coupled to a vacuum source. Accordingly, bodily fluids may be removed from the proximal end region 534 of the chamber 510 via the conduit 516.

The base 568, the sheath 570, the cap 578, and the conduit 516 may be attached together using any suitable method. For example, at least two of the base 568, the sheath 570, the cap 578, or the conduit 516 may be attached together using at least one of an interference fit, an adhesive, stitching, welding (e.g., ultrasonic welding), tape, any other suitable method, or combinations thereof.

In some examples (not shown), the fluid collection assembly 502 may have a one piece design, with one or more of the sheath 570, the base 568, and the cap 578 being a single, integrally formed piece.

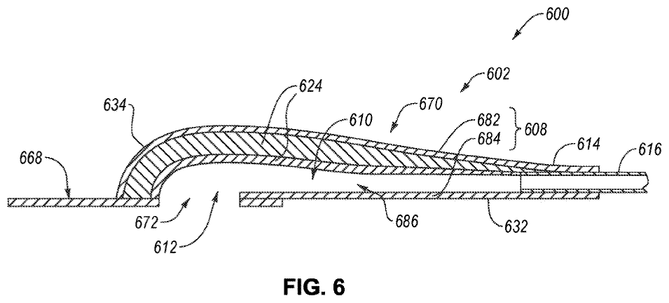

FIG. 6 is a cross-sectional view of a fluid collection assembly 602, according an embodiment. The fluid collection assembly 602 is an example of a male fluid collection assembly though, in some embodiments, the fluid collection assembly 602 may be used to receive bodily fluids from a female urethral opening. The fluid collection assembly 602 includes a sheath 670 and a base 668. The base 668 is configured to be attached (e.g., permanently attached to or configured to be permanently attached) to the sheath 670. The base 668 is also configured to be attached to the region about the urethral opening (e.g., penis) of the individual.

The sheath 670 includes a fluid impermeable barrier 608 that is at least partially formed from a first panel 682 and a second panel 684. The first panel 682 and the second panel 684 may be attached or integrally formed together (e.g., exhibits single piece construction). In an embodiment, as illustrated, the first panel 682 and the second panel 684 are distinct sheets. The fluid impermeable barrier 608 also defines a chamber 610 between the first panel 682 and the second panel 684, an opening 612 at a proximal end region 634 of the sheath 670, and a fluid outlet 614 at a distal end region 632 of the sheath 670. The sheath 670 also includes at least one porous material 624 disposed in the chamber 610.

The inner surface(s) of the fluid impermeable barrier 608 (e.g., inner surfaces of the first and second panels 682, 684 at least partially defines the chamber 610 within the fluid collection assembly 602. The fluid impermeable barrier 608 temporarily stores the bodily fluids in the chamber 610. The fluid impermeable barrier 608 may be formed from any of the fluid impermeable materials disclosed herein. As such, the fluid impermeable barrier 608 substantially prevents the bodily fluids from passing through the fluid impermeable barrier 608.

In an embodiment, at least one of the first panel 682 or the second panel 684 is formed from an at least partially transparent fluid impermeable material, such as polyethylene, polypropylene, polycarbonate, or polyvinyl chloride. Forming at least one of the first panel 682 or the second panel 684 from an at least partially transparent fluid impermeable material allows a person (e.g., medical practitioner) to examiner the penis. In some embodiments, both the first panel 682 and the second panel 684 are formed from at least partially transparent fluid impermeable material. Selecting at least one of the first panel 682 or the second panel 684 to be formed from an at least partially transparent impermeable material allows the penis to be examined without detaching the entire fluid collection assembly 602 from the region about the penis. For example, the chamber 610 may include a penis receiving area 686 that is configured to receive the penis of the individual when the penis extends into the chamber 610. The penis receiving area 686 may be defined by at least the porous material 624 and at least a portion of the at least partially transparent material of the first panel 682 and/or the second panel 684. In other words, the porous material 624 is positioned in the chamber 610 such that the porous material 624 is not positioned between the penis and at least a portion of the transparent portion of the first panel 682 and/or second panel 684 when the penis is inserted into the chamber 610 through the opening 612. The porous material 624 is generally not transparent and, thus, the portion of the at least partially transparent material of the first panel 682 and/or the second panel 684 that defines the penis receiving area 686 forms a window which allows the person to view into the penis receiving area 686 and examine the penis.

The opening 612 defined by the fluid impermeable barrier 608 provides an ingress route for bodily fluids to enter the chamber 610 when the penis is a buried penis and allow the penis to enter the chamber 610 (e.g., the penis receiving area 686) when the penis is not buried. The opening 612 may be defined by the fluid impermeable barrier 608 (e.g., an inner edge of the fluid impermeable barrier 608). For example, the opening 612 is formed in and extends through the fluid impermeable barrier 608 thereby enabling bodily fluids to enter the chamber 610 from outside of the fluid collection assembly 602.

The fluid impermeable barrier 608 defines the fluid outlet 614 sized to receive the conduit 616. The conduit 616 may be at least partially disposed in the chamber 610 or otherwise in fluid communication with the chamber 610 through the fluid outlet 614. The fluid outlet 614 may be sized and shaped to form an at least substantially fluid tight seal against the conduit 616 thereby substantially preventing the bodily fluids from escaping the chamber 610. In an embodiment, the fluid outlet 614 may be formed from a portion of the first panel 682 and the second panel 684 that are not attached or integrally formed together. In such an embodiment, the fluid impermeable barrier 608 may not include a cap exhibiting a rigidity that is greater than the portions of the fluid impermeable barrier 608 thereabout which may facilitate manufacturing of the fluid collection assembly 602 may decreasing the number of parts that are used to form the fluid collection assembly 602 and may decrease the time required to manufacture the fluid collection assembly 602. The lack of the cap may make securing the conduit 616 to the fluid outlet 614 using interference fit to be difficult though, it is noted, attaching the conduit 616 to the fluid outlet 614 may still be possible. As such, the conduit 616 may be attached to the fluid outlet 614 (e.g., to the first and second panels 682, 684) using an adhesive, a weld, or otherwise bonding the fluid outlet 614 to the fluid outlet 614. Attaching the conduit 616 to the fluid outlet 614 may prevent leaks and may prevent the conduit 616 from inadvertently becoming detached from the fluid outlet 614. In an example, the conduit 616 may be attached to the fluid outlet 614 in the same manufacturing step that attaches the first and second panels 682, 684 together.

As previously discussed, the sheath 670 includes at least one porous material 624 disclosed in the chamber 610. The porous material 624 may direct the bodily fluids to one or more selected regions of the chamber 610, such as away from the penis and towards the fluid outlet 614. The porous material 624 may be formed from any of the porous materials disclosed herein. In an example, the porous material 624 may be formed from a single layer, two layers (e.g., a fluid permeable membrane extending across the opening 612 and a fluid permeable support since the fluid permeable membrane may be formed from a relatively foldable, flimsy, or otherwise easily deformable material), or three or more layers. In an example, the porous material 624 may be formed from a nonwoven material or a woven material (e.g., spun nylon fibers). In an example, the porous material 624 may include at least one material exhibiting substantially no absorption or at least one absorbent or adsorbent material.

In an embodiment, the porous material 624 may be a sheet. Forming the porous material 624 as a sheet may facilitate the manufacturing of the fluid collection assembly 602. For example, forming the porous material 624 as a sheet allows the first panel 682, the second panel 684, and the porous material 624 to each be sheets. During the manufacturing of the fluid collection assembly 602, the first panel 682, the second panel 684, and the porous material 624 may be stacked and then attached to each other in the same manufacturing step. For instance, the porous material 624 may exhibit a shape that is the same size or, more preferably, slightly smaller than the size of the first panel 682 and the second panel 684. As such, attaching the first panel 682 and the second panel 684 together along the outer edges thereof may also attach the porous material 624 to the first panel 682 and the second panel 684. The porous material 624 may be slightly smaller than the first panel 682 and the second panel 684 such that the first panel 682 and/or the second panel 684 extend around the porous material 624 such that the porous material 624 does not form a passageway through the fluid impermeable barrier 608 through which the bodily fluids may leak. Also, attaching the porous material 624 to the first panel 682 and/or the second panel 684 may prevent the porous material 624 from significantly moving in the chamber 610, such as preventing the porous material 624 from bunching together near the fluid outlet 614. In an example, the porous material 624 may be attached to the first panel 682 or the second panel 684 (e.g., via an adhesive) before or after attaching the first panel 682 to the second panel 684. In an example, the porous material 624 may merely be disposed in the chamber 610 without attaching the porous material 624 to at least one of the first panel 682 or the second panel 684. In an embodiment, the porous material 624 may exhibit shapes other than a sheet, such as a hollow generally cylindrical shape.

Generally, the sheath 670 is substantially flat when the penis is not in the penis receiving area 686 and the sheath 670 is resting on a flat surface. The sheath 670 is substantially flat because the fluid impermeable barrier 608 is formed from the first panel 682 and the second panel 684 instead of a generally tubular fluid impermeable barrier. Further, as previously discussed, the porous material 624 may be a sheet, which also causes the sheath 670 to be substantially flat. The sheath 670 may also be substantially flat because the fluid collection assembly 602 may not include relatively rigid rings or caps that exhibit a rigidity that is greater than the portions of the fluid impermeable barrier 608 thereabout since such rings and caps may inhibit the sheath 670 being substantially flat. It is noted that the sheath 670 is described as being substantially flat because at least one of the porous material 624 may cause a slight bulge to form in the sheath 670 depending on the thickness of the porous material 624, the fluid outlet 614 and/or conduit 616 may cause a bulge thereabout, or the base 668 may pull on portions of the sheath 670 thereabout. It is also noted that the sheath 670 may also be compliant and, as such, the sheath 670 may not be substantially flat during use since, during use, the sheath 670 may rest on a non-flat surface (e.g., may rest on the testicles, the perineum, and/or between the thighs) and the sheath 670 may conform to the surface of these shapes.

The ability of the sheath 670 to be substantially flat when the penis is not in the penis receiving area 686 and the sheath 670 is resting on a flat surface allows the fluid collection assembly 602 to be used with a buried and a non-buried penis. For example, when the fluid collection assembly 602 is being used with a buried penis, the penis does not extend into the penis receiving area 686 which causes the sheath 670 to lie relatively flat across the aperture 672 of the base 668. When the sheath 670 lies relatively flat across the aperture 672, the porous material 624 extends across the opening 612 and the aperature 672 and is in close proximity to the buried penis. As such, the porous material 624 prevents or inhibits pooling of bodily fluids discharged from the buried penis against the skin of the individual since the porous material 624 will receive and remove at least a significant portion of the bodily fluids that would otherwise pool against the skin of the individual. Thus, the skin of the individual remains dry thereby improving comfort of using the fluid collection assembly 602 and preventing skin degradation. However, unlike other conventional fluid collection assemblies that are configured to be used with buried penises, the fluid collection assembly 602 may still be used with a non-buried penis since the non-buried penis can still be received into the penis receiving area 686, even when the penis is fully erect. Additionally, the ability of the sheath 670 to be substantially flat allows the fluid collection assembly 602 to be used more discretely than if the sheath 670 was not substantially flat thereby avoiding possibly embarrassing scenarios.

When the sheath 670 is substantially flat, the porous material 624 occupies substantially all of the chamber 610 and the penis receiving area 686 is collapsed (shown as being non-collapsed in FIG. 6 for illustrative purposes to show the penis receiving area 686). In other words, the sheath 670 may not define a region that is constantly unoccupied by the porous material 624. When the porous material 624 occupies substantially all of the chamber 610, the bodily fluids discharged into the chamber 610 are unlikely to pool for significant periods of time since pooling of the bodily fluids may cause sanitation issues, cause an odor, and/or may cause the skin of the individual to remain in contact with the bodily fluids which may cause discomfort and skin degradation.

As previously discussed, the first panel 682, the second panel 684, and the porous material 624 may be selected to be relatively flexible. The first panel 682, the second panel 684, and the porous material 624 are relatively flexible when the first panel 682, the second panel 684, and the porous material 624, respectively, are unable to maintain their shape when unsupported. The flexibility of the first panel 682, the second panel 684, and the porous material 624 may allow the sheath 670 to be substantially flat, as discussed above. The flexibility of the first panel 682, the second panel 684, and the porous material 624 may also allow the sheath 670 to conform to the shape of the penis even when the size and shape of the penis changes (e.g., becomes erect) and to minimize any unoccupied spaces in the chamber 610 in which bodily fluids may pool.

As previously discussed, the fluid collection assembly 602 includes a base 668 that is configured to be attached to the sheath 670. For example, the base 668 is configured to be permanently attached to the sheath 670. The base 668 is configured to be permanently attached to the sheath 670 when, for example, when the fluid collection assembly 602 is provided with the base 668 permanently attached to the sheath 670 or the base 668 is provided without being permanently attached to the sheath 670 but is configured to be permanently attached to the sheath 670 at some point in the future. Permanently attached means that the sheath 670 cannot be detached from the base 668 without damaging at least one of the sheath 670 or the base 668, using a blade to separate the sheath 670 from the base 668, and/or using chemicals to dissolve the adhesive that attaches the sheath 670 from the base 668. The base 668 may be permanently attached to the sheath 670 using an adhesive, sewing, heat sealing, RF welding, or US welding. In an embodiment, the base 668 is configured to be reversibly attached to the sheath 670. In an embodiment, the base 668 is integrally formed with the sheath 670.

The base 668 includes an aperture 672. The base 668 is permanently attached to the distal end region 632 of the sheath 670 such that the aperture 672 is aligned with the opening 612.

The base 668 is sized, shaped, and made of a material to be coupled to the skin that surrounds the penis (e.g., mons pubis, thighs, testicles, and/or perineum) and have the penis disposed therethrough. For example, the base 668 may define an aperture 672 configured to have the penis positioned therethrough. In an example, the base 668 may exhibit the general shape or contours of the skin surface that the base 668 is configured to be coupled with. The base 668 may be flexible, thereby allowing the base 668 to conform to any shape of the skin surface and mitigate the base 668 pulling the on skin surface. The base 668 may extend laterally past the sheath 670 thereby increasing the surface area of the skin of the individual to which the fluid collection assembly 602 may be attached compared to a substantially similar fluid collection assembly 602 that did not include a base.

As previously discussed, the fluid collection assembly 602 may include the conduit 616. The inlet of the conduit 616 may be located near the distal end region 632 of the sheath 670 which is expected to be the gravimetrically low point of the chamber 610 when worn by an individual. Locating the inlet at or near the distal end region 632 of the sheath 670 enables the conduit 616 to receive more of the bodily fluids than if the inlet of the conduit 616 was located elsewhere and reduce the likelihood of pooling (e.g., polling of the bodily fluids may cause microbe growth and foul odors). In an embodiment, the conduit 616 may be omitted, for example, when the fluid outlet 616 is directly attached to the fluid inlet of the fluid storage container (similar to what is shown in FIGS. 1A-2) or the port of the vacuum source (not shown).

Other examples of fluid collection assemblies that may be used in the fluid collection systems disclosed herein are disclosed in U.S. Pat. No. 10,973,678 filed on Jun. 2, 2017, U.S. Pat. No. 10,390,989 filed on Sep. 8, 2016, U.S. Pat. No. 10,226,376 filed on Jun. 3, 2017,.S. U.S. Pat. No. 10,952,889 filed on Jun. 2, 2016, International Publication No. WO2022/006256 published on Jan. 6, 2022, U.S. patent application Ser. No. 16/433,773 filed on Jun. 6, 2019, U.S. Provisional Patent Application No. 63/134,754 filed on Jan. 7, 2021, International Publication No. WO2021/016026 published on Jan. 28, 2021, International Publication No. WO2021/207621 published on Oct. 14, 2021, U.S. patent application Ser. No. 17/597,673 filed on Jun. 16, 2020, and U.S. Provisional Patent Application No. 63/208,262 filed on Jun. 8, 2021, the disclosure of each of which is incorporated herein, in its entirety, by this reference. In an embodiment, these fluid collection assembly may be directly attached to the fluid inlet of the fluid storage container (as shown in FIG. 2) or the port of the vacuum source. In an embodiment, these fluid collection assemblies may be include a conduit connecting these fluid collection assemblies to the fluid storage container, as shown in FIG. 3.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection system, comprising:
a fluid collection assembly including:
a fluid impermeable barrier at least defining a chamber, at least one opening, and a fluid outlet; and
at least one porous material disposed in the chamber;
a fluid storage container; and
a vacuum source;
wherein:
the fluid collection assembly, the fluid storage container, and the vacuum source are in fluid communication with each other such that at least some of one or more bodily fluids received into the chamber and removed from the chamber and disposed into the fluid storage container using a suction provided by the vacuum source; and
at least one of:
the fluid impermeable barrier is directly attachable to at least one of the fluid storage container or the vacuum source; or
the fluid collection system further comprises at least one conduit connected to the fluid outlet of the fluid collection assembly and one of the fluid storage container or the vacuum source, the at least one conduit exhibiting a length of about 25 cm or less.

2. The fluid collection system of claim 1, wherein the fluid impermeable barrier is directly attached to at least one of the fluid storage container or the vacuum source.

3. The fluid collection system of claim 2, wherein the fluid impermeable barrier directly contacts the fluid storage container.

4. The fluid collection system of claim 2, wherein the fluid impermeable barrier directly contacts the vacuum source.

5. The fluid collection system of claim 1, wherein the fluid collection system further comprises the at least one conduit connected to the fluid outlet of the fluid collection assembly and one of the fluid storage container or the vacuum source, the at least one conduit exhibiting a length of about 25 cm or less.

6. The fluid collection system of claim 1, wherein the fluid storage container and the vacuum source are configured to be at least partially positioned between at least one of a urethral opening of an individual and clothing of the individual or thighs of the individual when the opening of the fluid collection assembly is positioned adjacent to a urethral opening of the individual or receives a penis of the individual.

7. The fluid collection system of claim 1, wherein at least one of the fluid storage container or the vacuum source exhibit a shape having a generally triangular cross-sectional shape.

8. The fluid collection system of claim 1, wherein the fluid collection assembly and the fluid storage container are configured for single use.

9. The fluid collection system of claim 1, wherein the fluid storage container defines an enclosure exhibiting a volume of about 500 mL or less.

10. The fluid collection system of claim 1, further comprising at least one seal element configured to prevent bodily fluids leaking from the fluid storage container.

11. The fluid collection system of claim 1, wherein the fluid storage container is positioned upstream from the vacuum source.

12. The fluid collection system of claim 1, wherein the vacuum source is positioned upstream from the fluid storage container.

13. A method of using a fluid collection system, the method comprising:
positioning at least one opening of a fluid collection assembly to be adjacent to a urethral opening of an individual or have a penis of the individual disposed through the at least one opening, the fluid collection assembly including a fluid impermeable barrier defining at least a chamber, the at least one opening, and the fluid outlet, the fluid collection assembly including at least one porous material disposed in the chamber;
receiving one or more bodily fluids from the individual into the chamber and into the at least one porous material;
applying a suction to the chamber via a vacuum source to remove at least some of the bodily fluids in the chamber and deposit the bodily fluids in a fluid storage container;
wherein at least one of:
the fluid impermeable barrier is directly attached to at least one of the fluid storage container or the vacuum source; or
the fluid collection system further comprises at least one conduit connected to the fluid outlet of the fluid collection assembly and one of the fluid storage container or the vacuum source, the at least one conduit exhibiting a length of about 25 cm or less.

14. The method of claim 13, wherein the fluid impermeable barrier is directly attached to at least one of the fluid storage container or the vacuum source.

15. The method of claim 13, wherein the fluid collection system further comprises at least one conduit connected to the fluid outlet of the fluid collection assembly and one of the fluid storage container or the vacuum source, the at least one conduit exhibiting a length of about 25 cm or less.

16. The method of claim 13, wherein positioning the at least one opening of the fluid collection assembly to be adjacent to the urethral opening of the individual or have a penis of the individual disposed through the at least one opening and receiving one or more bodily fluids from the individual into the chamber and into the at least one porous material is performed while the individual is seated.

17. The method of claim 13, wherein positioning the at least one opening of the fluid collection assembly to be adjacent to the urethral opening of the individual or have a penis of the individual disposed through the at least one opening, receiving one or more bodily fluids from the individual into the chamber and into the at least one porous material, and applying a suction to the chamber via a vacuum source is performed substantially without removing clothing from the individual.

18. The method of claim 13, wherein positioning the at least one opening of the fluid collection assembly to be adjacent to the urethral opening of the individual or have a penis of the individual disposed through the at least one opening includes positioning at least a portion of the fluid storage container and the vacuum source between the urethral opening of the individual and clothing of the individual.

19. The method of claim 13, wherein positioning the at least one opening of the fluid collection assembly to be adjacent to the urethral opening of the individual or have a penis of the individual disposed through the at least one opening includes positioning at least a portion of the fluid storage container and the vacuum source between thighs of the individual.

20. The method of claim 13, further comprising, after applying a suction to the chamber via a vacuum source to remove at least some of the bodily fluids in the chamber and deposit the bodily fluids in a fluid storage container, detaching the fluid collection assembly from the vacuum source and disposing of the fluid collection assembly.

21. The method of claim 13, further comprising, after applying a suction to the chamber via a vacuum source to remove at least some of the bodily fluids in the chamber and deposit the bodily fluids in a fluid storage container, detaching the fluid storage container from the vacuum source and disposing of the fluid storage container.

22. The method of claim 13, further comprising, after applying a suction to the chamber via a vacuum source to remove at least some of the bodily fluids in the chamber and deposit the bodily fluids in a fluid storage container:
removing the one or more bodily fluids from the fluid storage container; and
cleaning the fluid storage container.
